(19) United States
(12) Patent Application Publication
(10) Pub. No.: US 2023/0140758 A1
(43) Pub. Date: May 4, 2023

(54) GUIDEWIRE SIZING INSTRUMENTS, SYSTEMS, AND METHODS

(71) Applicant: Acumed LLC, Hillsboro, OR (US)

(72) Inventor: Brian R. Conley, Portland, OR (US)

(21) Appl. No.: 17/970,801

(22) Filed: Oct. 21, 2022

Related U.S. Application Data
(60) Provisional application No. 63/272,915, filed on Oct. 28, 2021.

(51) Int. Cl. A61B 90/00; A61B 5/107; A61B 17/00

(52) U.S. Cl. CPC A61B 90/06; A61B 5/1076; A61B 17/00234; A61B 2090/061

(57) ABSTRACT

A guidewire sizer that is integrated with a medical instrument the surgeon already uses in a surgical procedure involving a guidewire, or with another suitable item used in the surgical procedure is provided. The surgeon may, in some cases, use a single instrument for both measuring a guidewire diameter and performing at least one other task in a surgical procedure. The provided guidewire sizer may be a channel integrated with a body of a medical component, such as an instrument or other suitable item. The channel may be an indentation in the body's exterior surface or may be enclosed within the body. The channel includes at least two discrete portions that are each formed with a respective width that conforms to a dimension of a guidewire of a particular diameter, which enables measuring a guidewire diameter.

### PRIORITY CLAIM

This application claims priority to U.S. Provisional Patent Application No. 63/272,915 filed on Oct. 28, 2021, the entire contents of which are hereby incorporated by reference and relied upon.

### BACKGROUND

Surgical procedures sometimes require the use of a guidewire, such as a Kirschner-wire, or k-wire. In such procedures, a surgeon may need to select a guidewire of a particular diameter out of a set of guidewires having different diameters prior to inserting the guidewire into bone. For example, many screw systems contain different diameter screws that use different diameter guidewires for installation. As such, a surgeon must use a correctly sized guidewire for a particularly sized screw for proper installation. It can be difficult, however, to determine a guidewire size based solely on visual inspection of the guidewires. Typically, the surgeon may use a guidewire sizing instrument that is constructed as a plate with multiple holes of different sizes. The surgeon may insert a selected guidewire into one or more of the holes in the plate to find the particular hole that conforms to the guidewire. A marking on the plate indicates that particular hole's diameter, which is therefore the selected guidewire's diameter.

These typical guidewire sizing instruments, however, can be cumbersome for surgeons. In some instances, the typical guidewire sizing instrument may be attached as a bracket on a sterilization tray. In such instances, a surgeon has to hand a guidewire to an assistant, have them measure it, and hand it back to the surgeon, which adds time to a surgical procedure. In other instances, the typical guidewire sizing instrument can be its own standalone instrument (e.g., a plate) apart from the other instruments the surgeon may be using during a surgical procedure, which adds to the number of instruments in the operating room, such as on a sterilization tray. The surgeon also often, if not always, has to place another instrument down in order to use the typical guidewire sizing instrument, whether attached to a sterilization tray or a standalone instrument, which adds time to a surgical procedure. Additionally, the typical design of multiple holes in a plate having different diameters for guidewire sizing cannot be easily integrated with other instruments that the surgeon uses.

### SUMMARY

The present disclosure provides a new and innovative guidewire sizer that can be used for measuring a guidewire's diameter. The provided guidewire sizer may be integrated with a medical instrument or other suitable item used in a medical procedure such as a sterilization tray. As such, the provided guidewire sizer, in some cases, can help eliminate an instrument needed in the operating room, which can help reduce surgical procedure times. For instance, the guidewire sizer may be integrated with a medical instrument that a surgeon already uses for a separate task in a surgical procedure involving a guidewire, or the suitable item may be one that is already used in the medical procedure. In an example, a medical component used in a surgical procedure includes a body portion and a channel formed in the body portion. The channel includes a first portion adjacent a second portion, the first portion including an opening of the channel. The first portion of the channel has a first width sized to conform to a dimension of a first guidewire having a first diameter. The second portion of the channel has a second width sized to conform to a dimension of a second guidewire having a second diameter. The first diameter is greater than the second diameter.

In an aspect of the present disclosure, which may be combined with any other aspect listed herein unless specified otherwise, the medical component is a medical instrument used to perform a task in the surgical procedure.

In an aspect of the present disclosure, which may be combined with any other aspect listed herein unless specified otherwise, the medical component is one of a screw sizer, a handle of a driver, a targeting guide, a drill guide, a cannula, a handle of a cannula, a wire guide, a clamp, a retractor, a sterilization tray, or a screw caddy.

In an aspect of the present disclosure, which may be combined with any other aspect listed herein unless specified otherwise, the first width is equal to the first diameter.

In an aspect of the present disclosure, which may be combined with any other aspect listed herein unless specified otherwise, the second width is equal to the second diameter.

In an aspect of the present disclosure, which may be combined with any other aspect listed herein unless specified otherwise, the channel is an indentation on an exterior of the body portion.

In an aspect of the present disclosure, which may be combined with any other aspect listed herein unless specified otherwise, the channel is enclosed within the body portion.

In an aspect of the present disclosure, which may be combined with any other aspect listed herein unless specified otherwise, the channel further includes a third portion adjacent the second portion, the third portion of the channel having a third width sized to conform to a dimension of a third guidewire having a third diameter, and wherein the second diameter is greater than the third diameter.

In an aspect of the present disclosure, which may be combined with any other aspect listed herein unless specified otherwise, the third width is equal to the third diameter.

In an aspect of the present disclosure, which may be combined with any other aspect listed herein unless specified otherwise, the channel further includes a fourth portion adjacent the third portion, the fourth portion of the channel sized to conform to a dimension of a fourth guidewire having a fourth diameter, and wherein the third diameter is greater than the fourth diameter.

In an aspect of the present disclosure, which may be combined with any other aspect listed herein unless specified otherwise, the first portion of the channel includes a corner that extends a length of the first portion of the channel.

In another example, a system for obtaining a measurement of a guidewire includes a guidewire and a medical component. The guidewire has one of a first diameter or a second diameter. The medical component includes a body portion and a channel formed in the body portion. The channel includes a first portion adjacent a second portion, the first portion including an opening of the channel. The first portion of the channel has a first width sized to conform to a dimension of the guidewire having the first diameter. The second portion of the channel has a second width sized to conform to a dimension of the guidewire having the second diameter. The first diameter is greater than the second diameter.

In an aspect of the present disclosure, which may be combined with any other aspect listed herein unless specified otherwise, the guidewire has one of the first diameter, the second diameter, or a third diameter, wherein the channel of the medical component further includes a third portion adjacent the second portion, the third portion of the channel having a third width sized to conform to a dimension of the guidewire having the third diameter, and wherein the second diameter is greater than the third diameter.

In an aspect of the present disclosure, which may be combined with any other aspect listed herein unless specified otherwise, the guidewire has one of the first diameter, the second diameter, the third diameter, or a fourth diameter, wherein the channel further includes a fourth portion adjacent the third portion, the fourth portion of the channel having a fourth width sized to conform to a dimension of the guidewire having the fourth diameter, and wherein the third diameter is greater than the fourth diameter.

In another example, a method is provided for obtaining a measurement of a guidewire having one of a first diameter or a second diameter using a medical component. The medical component includes a body portion and a channel formed in the body portion. The channel includes a first portion adjacent a second portion, the first portion including an opening of the channel. The first portion of the channel is sized to conform to a dimension of the guidewire having the first diameter. The second portion of the channel is sized to conform to a dimension of the guidewire having the second diameter. The first diameter is greater than the second diameter. The method includes inserting a tip of the guidewire into the channel through the opening until advancement of the tip of the guidewire further into the channel is prevented. A measurement of the guidewire is then obtained.

In an aspect of the present disclosure, which may be combined with any other aspect listed herein unless specified otherwise, advancement of the tip of the guidewire further into the channel is prevented upon the tip of the guidewire reaching an endpoint of the second portion of the channel, and wherein the measurement of the guidewire is obtained as the second diameter.

In an aspect of the present disclosure, which may be combined with any other aspect listed herein unless specified otherwise, advancement of the tip of the guidewire further into the channel is prevented upon the tip of the guidewire reaching an endpoint of the first portion of the channel, and wherein the measurement of the guidewire is obtained as the first diameter.

In an aspect of the present disclosure, which may be combined with any other aspect listed herein unless specified otherwise, the guidewire has one of the first diameter, the second diameter, or a third diameter, wherein the channel of the medical component further includes a third portion adjacent the second portion, the third portion of the channel sized to conform to a dimension of the guidewire having the third diameter, and wherein the second diameter is greater than the third diameter.

In an aspect of the present disclosure, which may be combined with any other aspect listed herein unless specified otherwise, the guidewire has one of the first diameter, the second diameter, the third diameter, or a fourth diameter, wherein the channel further includes a fourth portion adjacent the third portion, the fourth portion of the channel sized to conform to a dimension of the guidewire having the fourth diameter, and wherein the third diameter is greater than the fourth diameter.

In an aspect of the present disclosure, which may be combined with any other aspect listed herein unless specified otherwise, the method further comprises performing a surgical procedural task using the medical component, the surgical procedural task being separate from obtaining the measurement of the guidewire.

Additional features and advantages of the disclosed method and apparatus are described in, and will be apparent from, the following Detailed Description and the Figures. The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the inventive subject matter.

### BRIEF DESCRIPTION OF THE DRAWINGS

FIG. 1 illustrates a prior art guidewire sizing instrument.

FIG. 2 illustrates a perspective view of a medical instrument having an integrated guidewire sizer, according to an aspect of the present disclosure.

FIGS. 3A to 3E illustrate magnified views of a guidewire measured using the integrated guidewire sizer of the medical instrument of FIG. 2, according to an aspect of the present disclosure.

### DETAILED DESCRIPTION

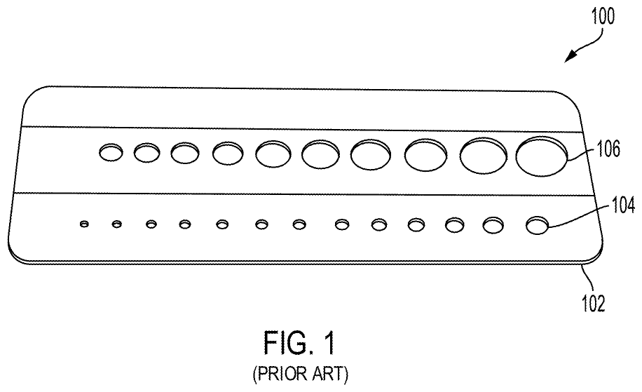

The present application provides a new and innovative guidewire sizer that can be used for measuring a guidewire's diameter. The provided guidewire sizer may be integrated with a medical instrument or other suitable item used in a medical procedure such as a sterilization tray. A surgeon typically has to use a separate guidewire sizing instrument to measure a guidewire's diameter. FIG. 1 illustrates an example typical guidewire sizing instrument 100 known in the art. The guidewire sizing instrument 100 includes a plate 102 and multiple holes 104 having different diameters in the plate 102. A surgeon may insert a guidewire through one or more of the holes 104 to find the hole 104 that conforms to the guidewire. A guidewire conforms to a hole 104 when there is little to no space between the guidewire's outer surface and the surface of the hole 104 when the guidewire is inserted through the hole 104. A marking on the plate 102 indicates to the surgeon a diameter of the conforming hole 104, which is therefore a diameter of the guidewire. In some instances, the typical guidewire sizing instrument 100 may include multiple holes 106 having different diameters for measuring a diameter of a different component, such as a nail.

The typical guidewire sizing instrument 100, however, can be cumbersome for surgeons because it is another instrument the surgeon has to use during a surgical procedure. The guidewire sizing instrument 100 therefore adds to the number of instruments in the operating room, such as on a sterilization tray. The surgeon also usually has to put down an instrument in order to use the typical guidewire sizing instrument 100, which adds time to a surgical procedure. Further, the plate 102 having the multiple holes 104 of different diameters cannot be easily integrated with other instruments that the surgeon uses.

The present disclosure helps solve the above drawbacks of the typical guidewire sizing instrument 100 by providing a guidewire sizer that is integrated with a medical instrument the surgeon already uses in a surgical procedure involving a guidewire, or with another suitable item used in a surgical procedure. For instance, the provided guidewire sizer may be integrated with a body of a medical instrument, such as with the body's exterior surface. The surgeon may therefore use a single instrument for both measuring a guidewire diameter and performing at least one other task in a surgical procedure, which thereby reduces the number of instruments in the operating room (e.g., on a sterilization tray) and the surgical procedure time (e.g., by eliminating the need for using a second instrument). Stated differently, in such instances, the surgeon does not have to interact with a sterilization tray, or grab an instrument from the sterilization tray, in order to measure a guidewire diameter, but rather has the tool to do so in the surgeon's hands when using the provided medical instrument with the integrated guidewire sizer. In other examples, the provided guidewire sizer may be integrated with another suitable item in the medical procedure, such as the sterilization tray, which still reduces the number of instruments in the operating room.

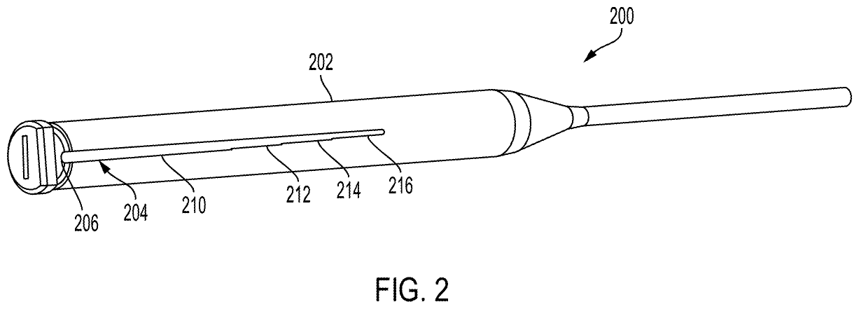

FIG. 2 illustrates a perspective view of an example medical instrument 200 having an integrated guidewire sizer. The medical instrument 200 may be any suitable instrument that a surgeon may use during a procedure in which a guidewire's diameter may need to be measured. For example, the medical instrument 200 may be screw sizers, driver handles, targeting guides, drill guides, cannulas, cannula handles, wire guides, clamps, retractors, etc. While the provided guidewire sizer is shown integrated with the medical instrument 200 for illustrative purposes, in other examples, the guidewire sizer may be integrated with another suitable item in a medical procedure. For example, the guidewire sizer may be integrated with a sterilization tray, a screw caddy, etc.

The example medical instrument 200 includes a body portion 202. A channel 204 having an opening 206 may be formed in the body portion 202. In some aspects, such as the one illustrated, the channel 204 may be an indentation in an outer surface of the body portion 202. In such aspects, only a portion of a guidewire's outer circumference (e.g., half of the outer circumference) contacts the body portion 202 when the guidewire is inserted into and advanced through the channel 204. In other aspects, the channel 204 may be enclosed within the body portion 202. In such other aspects, a guidewire's entire outer circumference is within the channel 204 when the guidewire is inserted into and advanced through the channel 204. Additionally, in such other aspects, a section of the body portion 202 may be transparent so that a guidewire can be viewed within the channel 204.

The channel 204 may have at least two distinct portions that are formed such that each distinct portion has a respective width that conforms to a dimension of a guidewire having a different diameter. For example, each distinct portion of the channel 204 may have a different width that is equal to a different diameter of a guidewire. As used herein, a portion of the channel 204 conforms to a guidewire if each side of the portion of the channel 204 defining a width of the portion of the channel 204 simultaneously contacts the guidewire.

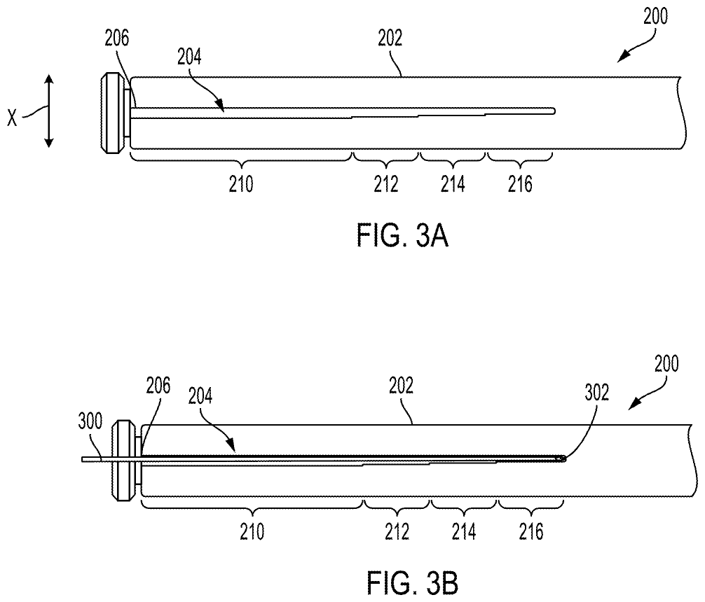

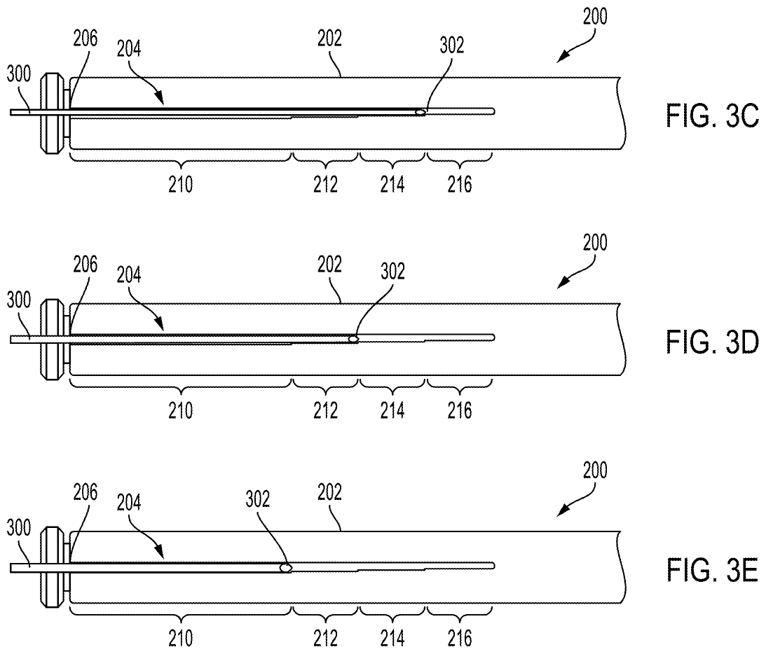

In the illustrated example, the channel 204 is shown having a first portion 210, a second portion 212, a third portion 214, and a fourth portion 216. The first portion 210 may be formed such that it has a width (e.g., in the direction X of FIG. 3A) that conforms to a dimension of a first guidewire having a first diameter (e.g., 1.4 millimeters). For example, the first portion 210 may have a width that is equal to the first diameter such that the width of the first portion 210 conforms to the first guidewire's diameter. In other examples, the first portion 210 may have a width that conforms to a chord of the first guidewire's circular cross-section other than the first guidewire's diameter such that the width of the first portion 210 is less than the first diameter. In such other examples, the respective width of each the second portion 212, the third portion 214, and the fourth portion 216 may similarly be sized to conform to an off-center chord of a guidewire such that each the first portion 210, the second portion 212, the third portion 214, and the fourth portion 216 still correspond to distinct guidewire sizes. In some aspects, the first portion 210 may have a curved surface. For example, the first portion 210 may be formed with a radius of curvature (e.g., 0.7 millimeters) that matches the first diameter of the first guidewire. In other aspects, the first portion 210 may have a non-curved surface. For example, the first portion 210 may be formed with a first side and second side meeting at a corner and a third side meeting the second side at a corner. Each corner may extend an entire length (e.g., perpendicular to the width) of the first portion 210.

The above description of the first portion 210 applies equally to the second portion 212, the third portion 214, and the fourth portion 216 except that each the second portion 212, the third portion 214, and the fourth portion 216 is sized for a different guidewire diameter. For instance, the second portion 212 may be formed such that it has a width that conforms to a dimension of a second guidewire having a second diameter (e.g., 1.1 millimeters) that is less than the first diameter. The third portion 214 may be formed such that it has a width that conforms to a dimension of a third guidewire having a third diameter (e.g., 0.9 millimeters) that is less than the second diameter. The fourth portion 216 may be formed such that it has a width that conforms to a dimension of a guidewire having a fourth diameter (e.g., 0.7 millimeters) that is less than the third diameter. In other examples, the channel 204 may include two, three, five, or another suitable quantity of discrete portions. For instance, if a surgical system with which the medical instrument 200 is used includes a set of three guidewires having different diameters to select from, then the channel 204 may include three discrete portions, one for each guidewire diameter in the set.

FIG. 3A illustrates a magnified view of the integrated guidewire sizer of the example medical instrument 200 showing the channel 204 and its discrete first portion 210, second portion 212, third portion 214, and fourth portion 216. To measure a guidewire using the integrated guidewire sizer of the medical instrument 200, a surgeon may insert a tip of the guidewire into the opening 206 of the channel 204 and advance the tip through the channel 204 until it can be advanced no further. At this point, the surgeon can obtain a diameter of the guidewire by reading a marking on the body portion 202 that corresponds to the portion of the channel past which the guidewire could not be advanced.

For example, FIG. 3B illustrates a guidewire 300 inserted through the channel 204. In this example, a surgeon was able to advance a tip 302 of the guidewire 300 through each of the first portion 210, the second portion 212, and the third portion 214, and to an endpoint of the fourth portion 216. As such, the diameter of the guidewire 300, in this example, is equal to the marking (“0.7 mm”) corresponding to the fourth portion 216.

In another example, FIG. 3C illustrates the guidewire 300 inserted through the channel 204 such that the surgeon was able to advance the tip 302 of the guidewire 300 through each of the first portion 210 and the second portion 212, and to an endpoint of the third portion 214. This is because, in this example, the diameter of the guidewire 300 is larger than a diameter for which the fourth portion 216 was formed, so the guidewire 300 cannot fit into the fourth portion 216 of the channel 204. As such, the diameter of the guidewire 300, in this example, is equal to the marking (“0.9 mm”) corresponding to the third portion 214.

In another example, FIG. 3D illustrates the guidewire 300 inserted through the channel 204 such that the surgeon was able to advance the tip 302 of the guidewire 300 through the first portion 210 and to an endpoint of the second portion 212. This is because, in this example, the diameter of the guidewire 300 is larger than a diameter for which the third portion 214 was formed, so the guidewire 300 cannot fit into the third portion 214 of the channel 204. As such, the diameter of the guidewire 300, in this example, is equal to the marking (“1.1 mm”) corresponding to the second portion 212.

In another example, FIG. 3E illustrates the guidewire 300 inserted through the channel 204 such that the surgeon was able to advance the tip 302 of the guidewire 300 to an endpoint of the first portion 210. This is because, in this example, the diameter of the guidewire 300 is larger than a diameter for which the second portion 212 was formed, so the guidewire 300 cannot fit into the second portion 212 of the channel 204. As such, the diameter of the guidewire 300, in this example, is equal to the marking (“1.4 mm”) corresponding to the first portion 210. The examples of FIGS. 3B to 3E therefore show how a surgeon can quickly and easily measure a guidewire diameter using the example medical instrument 200. The surgeon can then subsequently perform a separate surgical procedural task (e.g., determine a screw size for installation) using the medical instrument 200. In some examples, the surgeon may perform a separate surgical procedural task with the medical instrument 200 prior to using the medical instrument 200 to measure a guidewire diameter.

In the present disclosure, the terms “including” and “comprising” are used in an open-ended fashion, and thus should be interpreted to mean “including, but not limited to . . . .” As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

Reference throughout the specification to “various aspects,” “some aspects,” “some examples,” “other examples,” or “one aspect” means that a particular feature, structure, or characteristic described in connection with the aspect is included in at least one example. Thus, appearances of the phrases “in various aspects,” “in some aspects,” “certain embodiments,” “some examples,” “other examples,” “certain other embodiments,” or “in one aspect” in places throughout the specification are not necessarily all referring to the same aspect. Furthermore, the particular features, structures, or characteristics illustrated or described in connection with one example may be combined, in whole or in part, with features, structures, or characteristics of one or more other aspects without limitation.

It is to be understood that at least some of the figures and descriptions herein have been simplified to illustrate elements that are relevant for a clear understanding of the disclosure while eliminating, for purposes of clarity, other elements. Those of ordinary skill in the art will recognize, however, that these and other elements may be desirable. However, because such elements are well known in the art, and because they do not facilitate a better understanding of the disclosure, a discussion of such elements may not be provided herein.

The terminology used herein is intended to describe particular embodiments only and is not intended to be limiting of the present disclosure. As used herein, the singular forms “a,” “an,” and “the” are intended to include the plural forms as well, unless otherwise indicated. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term ‘at least one of X or Y’ or ‘at least one of X and Y’ should be interpreted as X, or Y, or X and Y.

Without further elaboration, it is believed that one skilled in the art can use the preceding description to utilize the claimed inventions to their fullest extent. The examples and aspects disclosed herein are to be construed as merely illustrative and not a limitation of the scope of the present disclosure in any way. It will be apparent to those having skill in the art that changes may be made to the details of the above-described examples without departing from the underlying principles discussed. In other words, various modifications and improvements of the examples specifically disclosed in the description above are within the scope of the appended claims. For instance, any suitable combination of features of the various examples described is contemplated.

The invention is claimed as follows:

1. A medical component used in a surgical procedure comprising:
a body portion; and
a channel formed in the body portion, the channel including a first portion adjacent a second portion, the first portion including an opening of the channel, wherein the first portion of the channel has a first width sized to conform to a dimension of a first guidewire having a first diameter, wherein the second portion of the channel has a second width sized to conform to a dimension of a second guidewire having a second diameter, and wherein the first diameter is greater than the second diameter.

2. The medical component of claim 1, wherein the medical component is a medical instrument used to perform a task in the surgical procedure.

3. The medical component of claim 1, wherein the medical component is one of a screw sizer, a handle of a driver, a targeting guide, a drill guide, a cannula, a handle of a cannula, a wire guide, a clamp, a retractor, a sterilization tray, or a screw caddy.

4. The medical component of claim 1, wherein the first width is equal to the first diameter.

5. The medical component of claim 1, wherein the second width is equal to the second diameter.

6. The medical component of claim 1, wherein the channel is an indentation on an exterior of the body portion.

7. The medical component of claim 1, wherein the channel is enclosed within the body portion.

8. The medical component of claim 1, wherein the channel further includes a third portion adjacent the second portion, the third portion of the channel having a third width sized to conform to a dimension of a third guidewire having a third diameter, and wherein the second diameter is greater than the third diameter.

9. The medical component of claim 8, wherein the third width is equal to the third diameter.

10. The medical component of claim 8, wherein the channel further includes a fourth portion adjacent the third portion, the fourth portion of the channel sized to conform to a dimension of a fourth guidewire having a fourth diameter, and wherein the third diameter is greater than the fourth diameter.

11. The medical component of claim 1, wherein the first portion of the channel includes a corner that extends a length of the first portion of the channel.

12. A system for obtaining a measurement of a guidewire, the system comprising:
a guidewire having one of a first diameter or a second diameter; and
a medical component including:
a body portion, and
a channel formed in the body portion, the channel including a first portion adjacent a second portion, the first portion including an opening of the channel, wherein the first portion of the channel has a first width sized to conform to a dimension of the guidewire having the first diameter, wherein the second portion of the channel has a second width sized to conform to a dimension of the guidewire having the second diameter, and wherein the first diameter is greater than the second diameter.

13. The system of claim 12, wherein the guidewire has one of the first diameter, the second diameter, or a third diameter, wherein the channel of the medical component further includes a third portion adjacent the second portion, the third portion of the channel having a third width sized to conform to a dimension of the guidewire having the third diameter, and wherein the second diameter is greater than the third diameter.

14. The system of claim 13, wherein the guidewire has one of the first diameter, the second diameter, the third diameter, or a fourth diameter, wherein the channel further includes a fourth portion adjacent the third portion, the fourth portion of the channel having a fourth width sized to conform to a dimension of the guidewire having the fourth diameter, and wherein the third diameter is greater than the fourth diameter.

15. A method for obtaining a measurement of a guidewire having one of a first diameter or a second diameter using a medical component including a body portion and a channel formed in the body portion, the channel including a first portion adjacent a second portion, the first portion including an opening of the channel, wherein the first portion of the channel is sized to conform to a dimension of the guidewire having the first diameter, wherein the second portion of the channel is sized to conform to a dimension of the guidewire having the second diameter, and wherein the first diameter is greater than the second diameter, the method comprising:
inserting a tip of the guidewire into the channel through the opening until advancement of the tip of the guidewire further into the channel is prevented; and
obtaining the measurement of the guidewire.

16. The method for obtaining the measurement of the guidewire of claim 15, wherein advancement of the tip of the guidewire further into the channel is prevented upon the tip of the guidewire reaching an endpoint of the second portion of the channel, and wherein the measurement of the guidewire is obtained as the second diameter.

17. The method for obtaining the measurement of the guidewire of claim 15, wherein advancement of the tip of the guidewire further into the channel is prevented upon the tip of the guidewire reaching an endpoint of the first portion of the channel, and wherein the measurement of the guidewire is obtained as the first diameter.

18. The method for obtaining the measurement of the guidewire of claim 15, wherein the guidewire has one of the first diameter, the second diameter, or a third diameter, wherein the channel of the medical component further includes a third portion adjacent the second portion, the third portion of the channel sized to conform to a dimension of the guidewire having the third diameter, and wherein the second diameter is greater than the third diameter.

19. The method for obtaining the measurement of the guidewire of claim 18, wherein the guidewire has one of the first diameter, the second diameter, the third diameter, or a fourth diameter, wherein the channel further includes a fourth portion adjacent the third portion, the fourth portion of the channel sized to conform to a dimension of the guidewire having the fourth diameter, and wherein the third diameter is greater than the fourth diameter.

20. The method for obtaining the measurement of the guidewire of claim 15, further comprising performing a surgical procedural task using the medical component, the surgical procedural task being separate from obtaining the measurement of the guidewire.
